(19) United States
(12) Patent Application Publication
(10) Pub. No.: US 2016/0089002 A1
(43) Pub. Date: Mar. 31, 2016

(54) A GAS AND LIQUID SUPPLY SYSTEM FOR AN ENDOSCOPE

(71) Applicant: PARTNERS FOR ENDOSCOPY LIMITED, Stoke on Trent, Staffordshire (GB)

(72) Inventors: Christian Burton, Staffordshire (GB); Robert Hartley, N/A (GB)

(21) Appl. No.: 14/891,791

(22) PCT Filed: May 19, 2014
(86) PCT No.: PCT/GB2014/051529

§ 371 Date: Nov. 17, 2015

(30) Foreign Application Priority Data
May 17, 2013 (GB) 1308976.8

(51) Int. Cl. A61B 1/00; A61B 1/12; A61B 1/015

(52) U.S. Cl. CPC A61B 1/00128; A61B 1/015; A61B 1/00119; A61B 1/00068; A61B 1/126

(57) ABSTRACT

A gas and liquid supply system for an endoscope comprises a first gas flow channel having a first end configured for connection to a gas supply source and a second end arranged to supply gas to the distal tip of an endoscope. A second gas flow channel is provided having a first end in fluid connection with and extending from the first gas flow channel between the first and second ends of the first gas flow channel and an opposing distal end. A liquid supply channel is also provided. A connector is arranged for connecting the first end of the liquid flow channel to a liquid source. The connector comprises a first connection port configured for connection to an end of the liquid supply channel, and a liquid source connection channel in fluid connection with the first port for connecting the first port to a liquid supply. A second connection port is arranged for connection to the distal end of the second gas flow channel. The first port and the liquid source connection channel are fluidly isolated from the second port to prevent fluid communication between the second port and the liquid source.

The present invention relates to a gas and liquid supply system for an endoscope, and in particular an air and water supply system.

An endoscope is a device for enabling a physician to look inside body cavity or organ of a patient in order to conduct an internal visual examination. An endoscope typically comprises an insertion part to be inserted inside a body cavity, which may be a rigid or flexible tube, and a hand held part to be held by an operator for performing various operations. An imaging device is mounted at the distal tip of the insertion part to capture an image of an object to be inspected, and a light delivery system is provided to illuminate the object of interest.

During an endoscopy bodily fluid or other matter within the body cavity may deposit on the imaging device at the distal tip of the insertion part. Similar fluid or matter within the cavity may obscure the view of the point of interest. An endoscope therefore typically includes an air and water delivery system arranged to deliver water to the distal tip to clean the imaging device and air to displace obscuring fluid or matter within the cavity.

A connection block connects the endoscope to a source of pressurised air and to a water reservoir which is typically a rigid water bottle or canister. The connection block is connected to the hand held part via a flexible tube referred to as an umbilicus within which a water channel and air channel extend. The hand held part contains a user operated valve arrangement for selectively allowing the flow or water or air to the distal tip via the insertion tube. A pressurised water supply is generated by diverting part of the pressurised air source into the water reservoir thereby raising the pressure within the reservoir. The pressurised air source comprises pressurised ambient air drawn from the surrounding atmosphere. The sterility of an atmospheric air source is of a satisfactory level to be channelled directly into a body cavity. However, during prolonged use, the concentration of airborne contaminants within the water reservoir, such as pathogens, bacteria and spores, has been found to reach unacceptable levels due to the continued passage of ambient air through the water.

It is therefore desirable to provide an improved gas and liquid supply system for an endoscope which addresses the above described problems, maintains a sterile water supply and/or which offers improvements generally.

According to the present invention there is provided a gas and liquid supply system for an endoscope as described in the accompanying claims.

In an embodiment of the invention there is provided a gas and liquid supply system for an endoscope comprising a first gas flow channel having a first end configured for connection to a gas supply source and a second end arranged to supply gas to the distal tip of an endoscope; a second gas flow channel having a first end in fluid connection with and extending from the first gas flow channel between the first and second ends of the first gas flow channel and an opposing distal end; a liquid supply channel; and a connector for connecting the first end of the liquid flow channel to a liquid source. The connector comprises a first connection port configured for connection to an end of the liquid supply channel, and a liquid source connection channel in fluid connection with the first port for connecting the first port to a liquid supply. A second connection port is arranged for connection to the distal end of the second gas flow channel. The first port and the liquid source connection channel are fluidly isolated from the second port to prevent fluid communication between the second port and the liquid source.

Fluidly isolating the second port from the first port and source connection channel, and hence preventing gas flow into the liquid source prevents contamination of the liquid source by ambient air from the compressed gas source. The dual port arrangement of the connector allows it to connect to the water and air connection port of a standard endoscope system and cause the air and water system of the endoscope to operate in a different manner. This advantageously allows the benefits of non-contamination of the water source, by completely isolating the water source from the airborne contaminants, to be achieved with a standard endoscope and without requiring entire system re-design or replacement.

The system may further comprise closure means for preventing gas flow through the second port. In the arrangement of the prior art the second port directs air flow into the liquid container. Pressurisation of the liquid container allows the air pressure at the control handle to be maintained as the pressure drop along the second gas flow channel is minimised once the liquid container reaches system pressure. In the arrangement of the present invention the air flow from the second channel is prevented from reaching the liquid container. Therefore, in order to maintain air supply pressure at the control handle the closure means blocks airflow out of the second gas flow channel.

Preferably the second port is configured to connect to and close the second gas flow channel. The second port comprises a closed channel which blanks out the second gas flow channel and a connection portion.

The system may further comprise a gas flow connection channel connected to the second port for allowing the supply of gas to the second gas flow channel via the second port, and valve means arranged to selectively close the gas supply connection channel. In a first configuration the valve means closes the second gas flow channel, allowing the pressurised air flow of the control unit to be operated without loss of system pressure at the control handle. In the open configuration the system may be operated without the pressurised air supply of the control unit, with a pressurised air or CO₂ supply being provided to the system via the second port. Preferably the valve means comprises a luer lock.

Preferably the liquid source comprises a liquid container connected to the liquid source connection channel and wherein the second port is fluidly isolated from the liquid container.

There may be provided means for pressurising the liquid container to cause liquid contained therein to flow from the liquid container to the first liquid flow channel via the first port. The pressurising means may be a mechanical and/or pneumatic means of pressurisation or any other suitable means. The water reservoir may comprise a flexible bag.

The connector may comprise body section having the first and second ports integrally formed therein. In this way the connector may be advantageously manufactured and provided as a single piece component thereby assisting manufacture and assembly.

In another aspect of the invention there is provided an endoscope comprising:
a control handle;
an insertion tube;
a connection block having a first gas supply port for connection to a pressurised air source, and second gas port and a water supply port for connection to a pressurised water source;
an umbilical tube connecting the connection block to the control handle;
a first gas flow channel extending from the connector block to the control handle via the umbilical cord having a first end connected to the gas supply port and a second end connected to the control handle;
a second gas flow channel branched from the first gas flow channel having a first end in fluid connection with and extending from the first gas flow channel and a second end connected to the second gas port; and
a liquid supply channel extending from the liquid supply port to the control handle via the umbilical tube;
a connector for connecting the liquid supply port to a liquid source, the connector comprising a first connection port configured for connection to the liquid supply channel, a liquid source connection channel in fluid connection with the first port for connecting the first port to a liquid source; and a second connection port configured for simultaneous connection to the second gas port, wherein the first connection port and the liquid source connection channel are fluidly isolated from the second connection port to prevent fluid communication between the second connection port and the liquid source.

In a further aspect of the invention there is provided a liquid supply system for an endoscope comprising:
a liquid receptacle, at least a portion of which is collapsible under the action of external pressure to pressurise the liquid contained therein;
an enclosure having an internal volume configured to house the liquid receptacle;
a fluid supply conduit in fluid communication with the internal volume of the enclosure for supplying a fluid thereto;
a liquid outlet conduit having a first end in fluid communication with the liquid receptacle and a second end for supplying liquid from the receptacle to an endoscope;
wherein the liquid receptacle and enclosure and arranged such that when a fluid is forced under pressure into the enclosure via the fluid supply conduit the resulting pressure rise within the enclosure causes pressurisation of the liquid contained within the receptacle.

In this way, the pressurised air source of an endoscope system may be used to pressurise the water source while maintaining the water and air in fluid isolation to prevent contamination of the water source. The sterility of the entire volume of the water may therefore be maintained throughout use. The use of the pressurised air source of the endoscope advantageously avoids utilises the existing source of pressurised air required for passing air to the distal tip, and avoids the need for a separate pressure source.

The liquid receptacle is a preferably flexible plastic bag. This allows the water to be provided in a sealed sterile form, with the bag allowing single use disposability. In contrast the bottle arrangements of the prior art require washing and sterilising after use at significantly increased cost.

The enclosure may comprise a closure member that is movable to an open configuration to permit the liquid receptacle to be inserted or removed from the enclosure. This enables the enclosure to be a permanent multi-use component that houses the single use disposable bags, with the closure member allowing access to insert and replace the bags. The closure member and/or the portion of the enclosure against which it closes preferably includes a seal.

The fluid supply conduit and the liquid outlet conduit are concentric and enter the enclosure through a common sealed port thereby only requiring a single port and single seal.

The fluid supply conduit and the liquid outlet conduit are fluidly isolated with the fluid supply conduit being open to the internal volume of the enclosure and the liquid outlet conduit being isolated from the internal volume of the enclosure and sealingly connected to the liquid receptacle thereby preventing contamination of the water source.

The liquid supply system may comprise a sealing member configured to seal and locate the fluid supply and liquid outlet conduits relative to the enclosure, wherein the sealing member comprises a fluid pathway in fluid communication with the fluid supply conduit and the internal volume of the enclosure for fluidly connecting the fluid supply conduit to the internal volume of the enclosure. The sealing member mat be a plug or cap or any other suitable means of securing to and sealing against the enclosure.

In a further aspect of the invention a liquid and fluid connection conduit for an endscope comprises:
a liquid conduit having a first end configured for connection to a liquid supply receptacle;
a concentric fluid conduit surrounding the liquid conduit and fluidly isolated therefrom having a first opening at an end corresponding to the first end of the liquid conduit;
a connector secured to the second ends of the liquid and fluid conduits, the connector comprising a first connection port configured for connection to the liquid supply channel, a liquid source connection channel in fluid connection with the first port for connecting the first port to a liquid source; and a second connection port configured for simultaneous connection to the second gas port, wherein the first connection port and the liquid source connection channel are fluidly isolated from the second connection port to prevent fluid communication between the second connection port and the liquid source.

The liquid and fluid connection conduit may comprise a second connector located at the opposing end of the conduits to the first connector for connecting the conduits to a pressurisation enclosure, the connector comprising a body through which the liquid conduit extends having and a liquid pathway fluidly connected to the fluid conduit opening to a distal surface of the body such that the fluid conduit is in open connection with the enclosure when the second connector is connected thereto.

The present invention will now be described by way of example only with reference to the following illustrative figures in which:

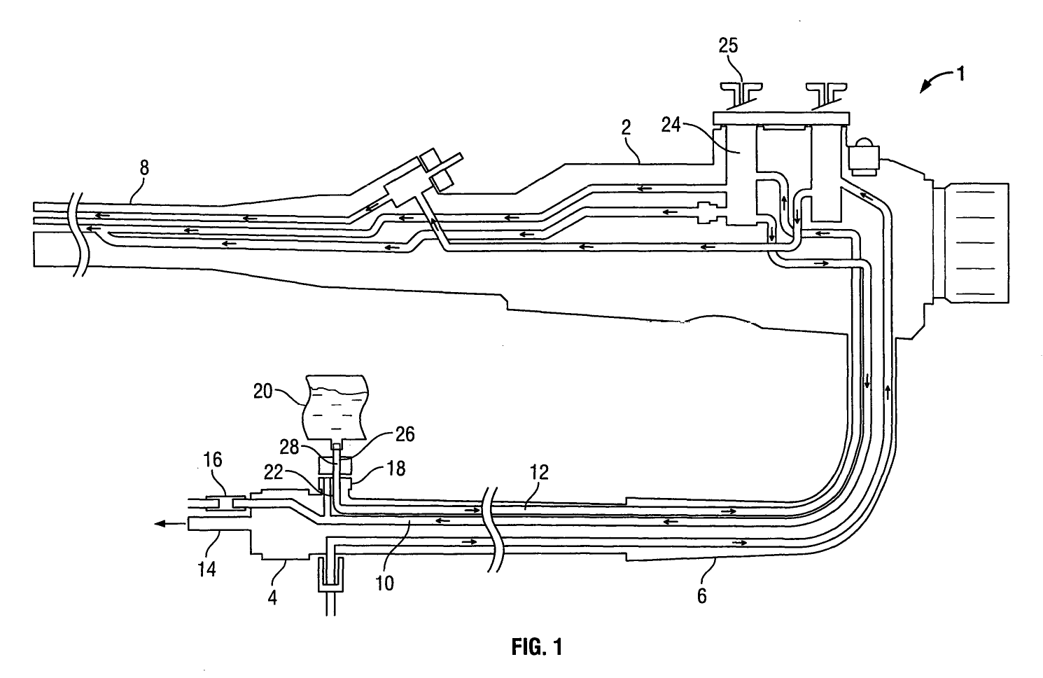

FIG. 1 shows a schematic diagram of an endoscope according to an embodiment of the present invention;

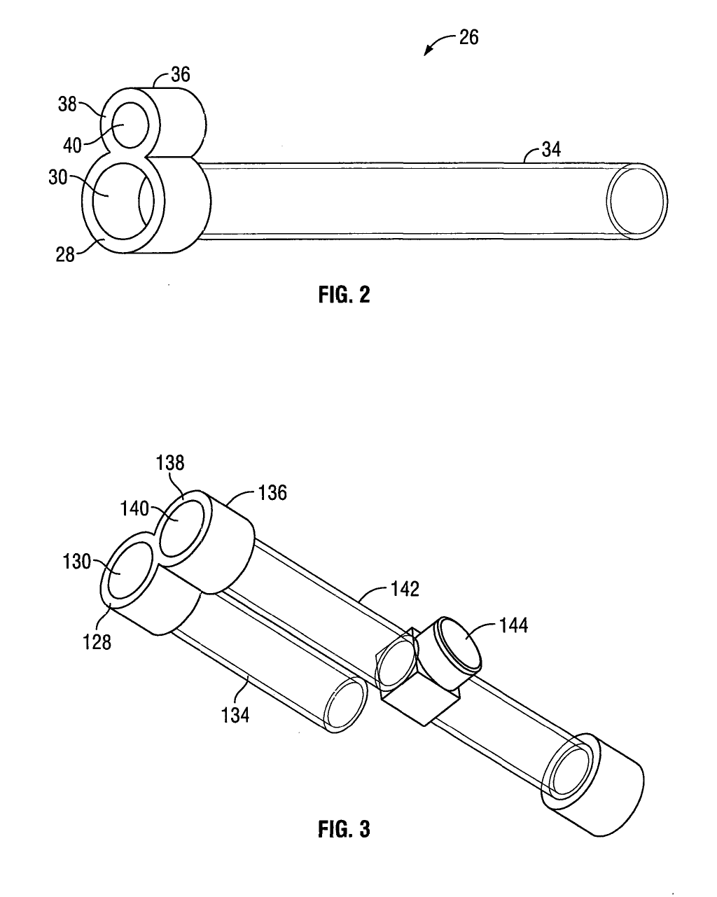

FIG. 2 shows an air and water connector according to an embodiment of the invention;

FIG. 3 shows an alternative air and water connector according to another embodiment of the invention;

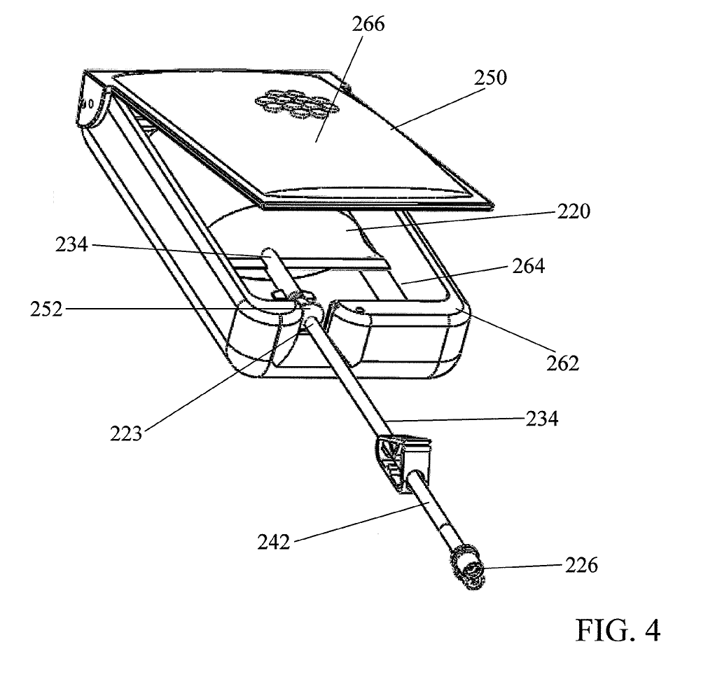

FIG. 4 shows a liquid pressurisation apparatus according to an embodiment of the invention;

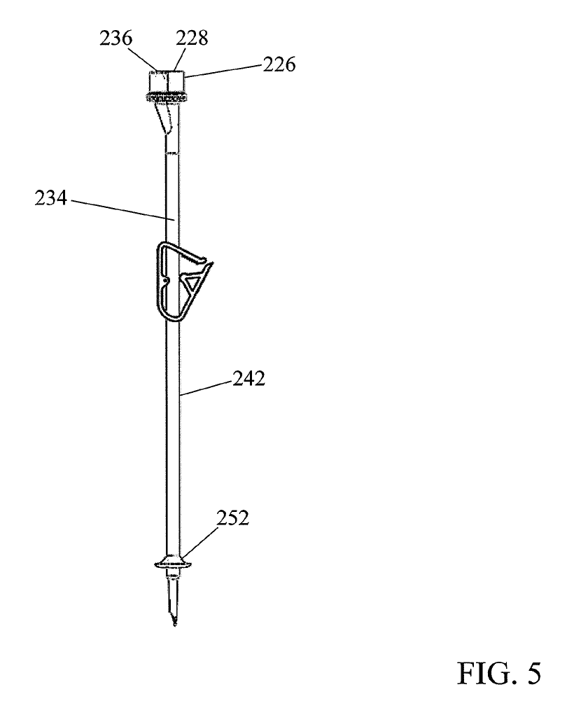

FIG. 5 shows a connection conduit according to an embodiment of the invention;

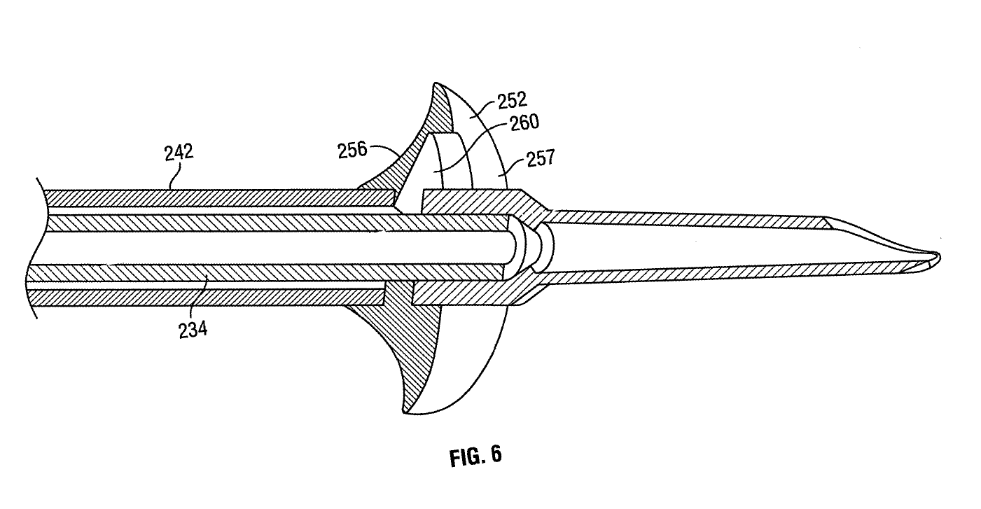

FIG. 6 shows a section view of the tip of the connector if FIG. 5 including a sealing plug for connection to an enclosure;

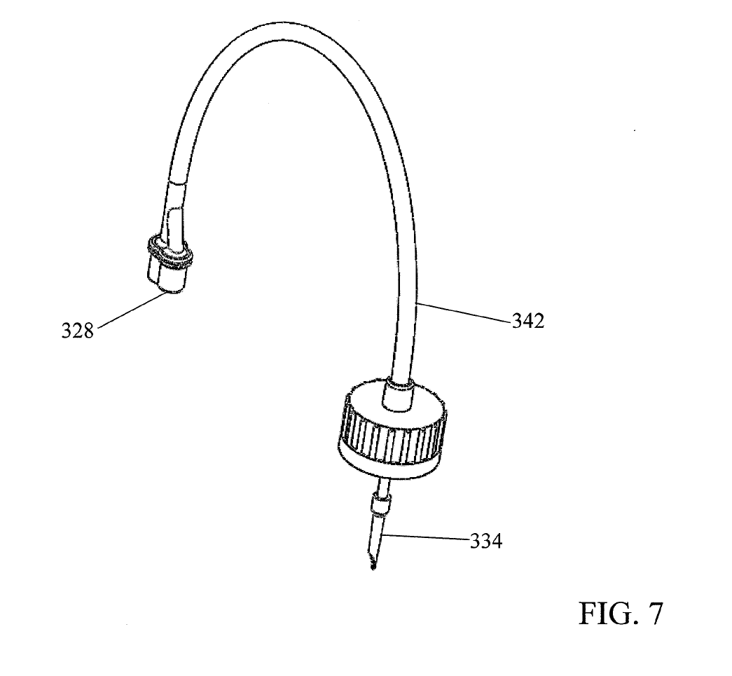

FIG. 7 shows an alternative connection conduit according to an embodiment of the invention; and

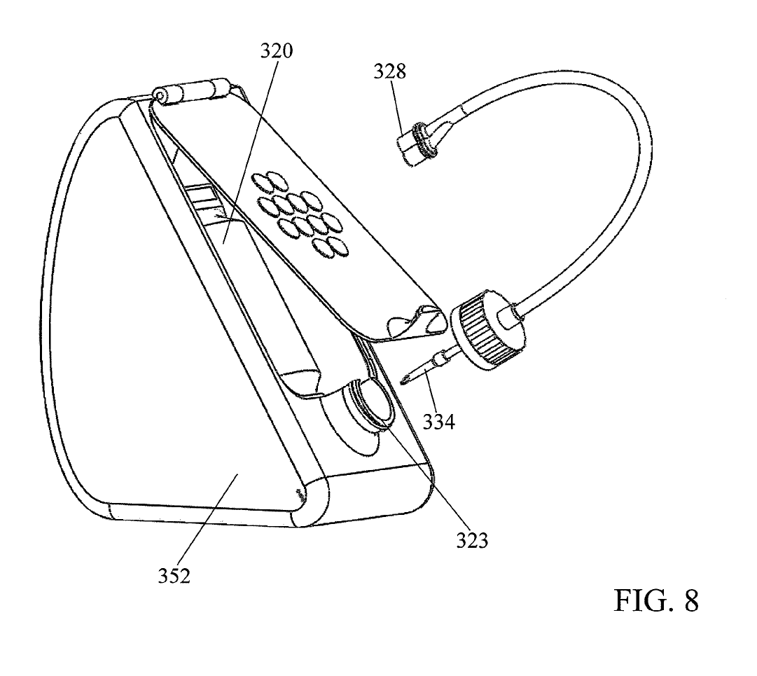

FIG. 8 shows the connection conduit of FIG. 7 in combination with a pressurisation enclosure.

Referring to FIG. 1 an endoscope 1 comprises a handheld control handle 2 that is held by the operator to control the endoscope and an insertion tube 8 for insertion into the patient connected to the distal end of the control handle 2. A flexible tube or umbilicus 6 is connected to the proximal end of the control handle 2 and defines a conduit connecting the control handle 2 to an air and water connection block 4.

The endoscope 1 includes an airflow channel 10 and a water channel 12 for providing air and water to the distal tip, and a fibre optic light cable (not shown). The connector 4 is configured to connect the endoscope 1 to a main control unit for the endoscope. The connection block 4 includes a light connector 14 which connects the fibre optic cable to a light source within the main control unit and an air connector 16 for connecting the air channel 10 to a pressurised air source within the main control unit. The air connector 16 is in fluid communication with the air channel 12 which extends through the connector block 4 and through the umbilicus 6 to the control handle 2. The connection block 4 also includes and air and water connection port 18.

A first end of the water channel 12 connects to the air and water port 18 for connection to a water reservoir 20. The air and water port 18 comprises a pair of spigot connectors extending from body of the connection block 4 and defining separately a water connection port 19 and an air connection port 21.

The air channel 10 is branched at a junction located within the connector 4 with a branched second air flow channel 22 extending from the main air channel 10 to the air and water port 18.

The control handle 2 comprises a valve assembly 24 arranged to control the flow of air and water to the distal tip of the insertion tube from the air channel 10 and water channel 12. The air channel 10 and water channel 12 are both fluidly connected to the valve 24 and in a first position pressurised air from the air channel 10 is vented to atmosphere via a central channel in the valve 24 which prevents airflow to the distal tip of the insertion tube, and the water channel 12 is closed preventing onward flow to the distal tip. By covering the central channel 25 within the valve this causes air to be diverted to the distal tip of the insertion tube while the flow of water to the distal remains closed. In a second position the valve is depressed and the flow of air to the distal tip is blocked while the water channel 12 is opened allowing the flow of liquid to the distal tip.

In arrangements to the prior art, a connector is secured to the air water connection including both air and water ports. An air channel extends from the air port and a water channel extends form the water port. The air channel extends internally inside the water channel, and both the air and water channels extend into the water reservoir container where the air channel and water channel fluidly connect. Within the connector block 4 the pressurised air flows splits along the air flow channel 10 with a portion of the air flow being diverted along the branch channel 22 and into the water reservoir via the air and water port. The flow of pressurised air into the water reservoir pressurises the water container thereby creating a pressurised water source. When the valve of the control handle is moved to the second position wherein the air flow is closed and the water channel 12 is open the flow of pressurised air into the water container forces the water out of the container along the water channel 12 to the distal tip of the insertion tube via the valve.

The pressurised air source connected to the air connector 16 includes a compressor which draws in ambient atmospheric air which is compressed and driven to the air channel 10. This atmospheric air contains contaminants present in the surrounding atmosphere. As the air is mixed with the water in the water reservoir these contaminants are absorbed into the water source. Over time the concentration of these contaminants builds up to an unacceptable level. It is therefore desirable to avoid the flow of atmospheric air into the water source as a means of pressurising the water source. However it is also desirable to avoid having to redesign and replace those endoscopes currently under manufacture and in use.

The present invention provides a connector 26 configured for connection to the air and water port 18 of the connection block 4 of a standard endoscope. The connector 26 includes a first water connection portion 28 comprising a substantially cylindrical body section with a channel 30 formed therethrough for connection to the water connection port 19. The water connection portion 28 includes an opening 32 at one end of the channel 30 configured for connection to the spigot water connection port 19 of the connector block 4. A water channel defined by a tube 34 extends from the opposing end of the channel 30 for connection to the water reservoir. The tube 34 may be inserted into or integrally formed with the connector 26. The connector 26 further includes an air connector portion 36 configured for connection to the air port 21 of the connector block 4.

In the embodiment shown in FIG. 2 the air connector portion 36 includes a body portion integrally formed with the water connection portion 28. The body portion is substantially cylindrical and includes a recess 38 configured to connect to the air port 21 of the connector block 4. The recess 38 includes a base 40 located at any predetermined depth into the body which covers and closes the air port 19 of the connector block 4 when connected thereto. The air connector section 36 is therefore a blanking connector which connects to and closes or plugs the air port 21. The air connector portion 36 also prevents air from entering the air channel 34 and therefore prevents air from entering the fluid reservoir 20, as well as ensuring that air pressure at the control handle 2 is maintained by preventing the free passage of air out of the air port 21.

Any suitable alternative means of pressurising the water reservoir 20 may be provided. The connector 26 enables an alternative pressurising means to be used with the standard endoscope system thereby avoids the use of ambient air from the air supply 16 for water pressurisation and without requiring a newly designed endoscope system having an alternative air and water supply system.

In an alternative arrangement shown in FIG. 3 the water connection portion 128 has a similar construction to the above described arrangement comprising a central body and a channel 130 connected to a tube 134 for connection to the water reservoir 120. The air connection portion 136 includes a cylindrical body and an opening 138 configured for connection to the air port 19 of the connector block 4. Rather than being a blanking connector the air connector 136 includes a channel 140 that extends through the connector but which is fluidly isolated from the channel 130 of the water connector 128. An air channel 142 extends from the opposing end of the air connector 136 and includes a locking valve 144 which is preferably a luer lock. The locking valve 144 selectively closes the air channel 142 thereby preventing air from exiting the air port 21 of the connector 4 from the second air channel 22 when the air channel 10 is connected to the pressurising air source of the main control panel via the connector 16.

In a second configuration the locking valve 144 may be opened to allow the supply of gas into the air port of the connector block 4 and into the air channel 22. Such a gas supply would be provided as an alternative to the pressurised air supply 16. The connector port for the pressurised air supply 16 connects to the main control unit simultaneously with the light source connector 14 and its distal end is located within the control unit in use.

Therefore, it is not possible to connect an alternative gas supply to this port 16 without requiring substantial reconfiguration of the main control unit and/or the connection block 4. The connector 126 enables an alternative supply of gas such as CO₂ to be provided into the endoscope system via the air port 21 of the connector block 4 which is not possible using the current system as this port is required for the outlet of air to the water reservoir. A CO₂ supply is preferable in certain circumstances to the pressurised air supply of the current system as it may be provided in a sterile form without contaminant and also provides benefit in that CO₂ is more readily absorbed into the body.

The air channel 142 extending from the air connector portion 136 is fluidly isolated from the water channel 134 such that air and water flow into and out of the air water port 18 of the connector block 4 are never mixed and are maintained in fluid isolation. Again in the second embodiment the connector 126 is configured for connection to a current endoscope system thereby obviating the requirement for system re-design and providing an economical means for achieving substantial system reconfiguration of the air water supply arrangement.

FIG. 4 shows a pressurisation apparatus 200 for providing pressurised water to an endoscope. The pressurisation assembly 200 comprises an enclosure 202 a sealed flexible liquid receptacle 220 which is preferably a sealed plastic bag of a similar material /.k, and construction to an IV bag. The enclosure 200 is reconfigurable between an open configuration as shown in Figure which it is able to receive the flexible bag 220, and a closed position. In the closed position a sealed volume is defined within the enclosure 202 in which the bag 220 is contained, with the internal volume of the enclosure being greater than the volume of the bag 220. The enclosure includes at least one port 223 for accommodating an air inlet and a water outlet. The enclosure is configured such that in the closed configuration the at least one port is sealed to prevent air escaping from the enclosure 202.

In the closed configuration pressurised air is passed into the enclosure 202 through the air inlet. The pressurised air surrounds at least part of the bag 220 within the enclosure. As the enclosure 202 is sealed the pressure with the enclosure rises as the pressurised air fills the enclosure 202. The pressurised air acts on the bag 202 to pressurise the liquid contents of the bag 202. When the liquid outlet is open the liquid is forced to flow out of the bag 202 under pressure due to the pressurisation of the enclosure 202.

FIG. 6 shows a connector 221 configured for use with the pressurisation apparatus 200. The connector 221 includes a proximal end connector 226 configured for connection to the air and water connection port of an endoscope. The proximal end connector 226 has the same construction as the connector 26 describes above, including a body portion having a water connection channel 228 and an air connection channel 236 defined therein. The water channel 228 and the air channel 236 are fluidly separate and isolated. The water channel 228 is connected to a water conduit 234 and the air channel 236 is connected to an air conduit 242. The water conduit 234 and air conduit 242 are concentric with the water conduit running longitudinally within and being surrounded by the air conduit 242, with the two conduits bifurcating at the proximal end to connect to the respective spaced channels 228 and 236.

At the distal end of the conduits a sealing plug 252 is provided. The sealing plug 252 is connected to the outer air conduit 242 with the water conduit extending through the sealing plug 252 to a greater longitudinal distance than the air conduit 242. The sealing member or plug 252 is configured to be received within and to close and seal the at least one port 223 of the enclosure 202 while permitting the water conduit 234 to extend into the enclosure 202 in a sealed manner. The distal end of the water conduit 234 includes a distal end connector 254 arranged to connect with a corresponding connection tube extending from the bag 220, with the end connector 254 having a tapered tip for piercing the bag 220 when connected thereto to fluidly connect the water conduit 234 to the bag in a sealed manner.

The sealing plug 252 includes a radially peripheral portion that seals against the at least one port 223 and a radially inner portion that seals against the air conduit 242. The proximal face 256 of the sealing plug 252 extends continuously between air conduit 242 and the port 223. The distal surface 257 includes at least one aperture 260 opening into the enclosure 202 and in fluid connection with the air conduit 242. The at least one aperture 260 allows air to flow from the air conduit 242 into the enclosure to pressurise the enclosure 202. The distal end of the water conduit 234 is sealed and connected to the water bag 220 and as such the water bag 220 is fluidly isolated from the enclosure 202 and from the air conduit 242. The air from the air conduit 234 is used to externally pressurise the bag 220 and hence pressure the water contained therein, but in contrast to the prior art the air is maintained in complete fluid isolation from the water within the bag 220 and as such the sterility of the water is maintained by preventing contact with the airborne contaminants.

The enclosure 202 includes a body section 262 comprising a plurality of walls defining an open chamber 264. A closure member in the form of a lid or closure flap 266 is pivotally connected to the body section 262 and pivots between an open position in which the chamber 264 is open and able to receive the bag 220, and a closed position in which the lid 266 closes and seals the chamber 262. It will be appreciated that the closure member 266 may be any other suitable means of opening and closing an opening to the enclosure 202 to permit insertion and removal of the bag 220, include a panel or door. Alternatively, the enclosure may be formed as a two-part clam shell arrangement with the two parts closing to form the sealed enclosure with either half defining the closure member.

Alternatively, the sealing member may be a sealing cap 352, as shown in FIG. 7. In this arrangement the cap 352 is a threaded cylindrical cap having a closed and is configured to threading engage or otherwise secure to a corresponding structure of the port 323 of the enclosure. The water conduit 334 and air conduit 342 extend through the cap 352, with the cap being secured to and sealed against the outer surface of the air conduit 342. The air conduit 342 terminates longitudinally before the water conduit 334 and is open to the internal volume of the cap 352. In this way, when the cap is connected to an enclosure, as shown in FIG. 8, the open end of the air conduit 342 is in open fluid communication with the internal volume of the enclosure 252 to allow pressurised air to be forced into the enclosure 352.

Whilst endeavouring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. A gas and liquid supply system for an endoscope comprising:
a first gas flow channel having a first end configured for connection to a gas supply source and a second end arranged to supply gas to the distal tip of an endoscope;
a second gas flow channel having a first end in fluid connection with and extending from the first gas flow channel between the first and second ends of the first gas flow channel and an opposing distal end;
a liquid supply channel; and
a connector for connecting the first end of the liquid flow channel to a liquid source, the connector comprising a first connection port configured for connection to an end of the liquid supply channel, a liquid source connection channel in fluid connection with the first port for connecting the first port to a liquid supply; and
a second connection port arranged for connection to the distal end of the second gas flow channel, wherein the first port and the liquid source connection channel are fluidly isolated from the second port to prevent fluid communication between the second port and the liquid source.

2. A gas and liquid supply system according to claim 1 further comprising closure means for preventing gas flow through the second port.

3. A gas and liquid supply system according to claim 2 wherein the second port is configured to connect to and close the second gas flow channel.

4. A gas and liquid supply system according to claim 3 wherein the second port comprises a closed channel.

5. A gas and liquid supply system according to claim 2 further comprising a gas flow connection channel connected to the second port for allowing the supply of gas to the second gas flow channel via the second port, and valve means arranged to selectively close the gas supply connection channel.

6. A gas and liquid supply system according to claim 5 wherein the valve means comprises a luer lock.

7. A gas and liquid delivery system according to claim 1 wherein the liquid source comprises a liquid container connected to the liquid source connection channel and wherein the second port is fluidly isolated from the liquid container.

8. A gas and liquid delivery system according to claim 7 further comprising means for pressurising the liquid container to cause liquid contained therein to flow from the liquid container to the first liquid flow channel via the first port.

9. A gas and liquid delivery system according to claim 1 wherein the connector comprises a body section having the first and second ports integrally formed therein.

10. An endoscope comprising:
a control handle;
an insertion tube;
a connection block having a first gas supply port for connection to a pressurised air source, and second gas port and a water supply port for connection to a pressurised water source;
an umbilical tube connecting the connection block to the control handle;
a first gas flow channel extending from the connector block to the control handle via the umbilical cord having a first end connected to the gas supply port and a second end connected to the control handle;
a second gas flow channel branched from the first gas flow channel having a first end in fluid connection with and extending from the first gas flow channel and a second end connected to the second gas port;
a liquid supply channel extending from the liquid supply port to the control handle via the umbilical tube;
a connector for connecting the liquid supply port to a liquid source, the connector comprising a first connection port configured for connection to the liquid supply channel, a liquid source connection channel in fluid connection with the first port for connecting the first port to a liquid source; and
a second connection port configured for simultaneous connection to the second gas port, wherein the first connection port and the liquid source connection channel are fluidly isolated from the second connection port to prevent fluid communication between the second connection port and the liquid source.

11. An endoscope according to claim 10 wherein the connector further comprises closure means for preventing gas flow through the second port.

12. An endoscope according to claim 11 wherein the second connection port is configured to connect to and close the second gas port to prevent gas flow through the second gas channel.

13. An endoscope according to claim 12 wherein the second connection port comprises a closed channel.

14. An endoscope according to claim 11 wherein the connector further comprises a gas flow connection channel connected to the second connection port for allowing the supply of gas to the second gas flow channel via the second gas port, and valve means arranged to selectively close the gas supply connection channel.

15. An endoscope according to claim 1 wherein the liquid source comprises a liquid container connected to the liquid source connection channel and wherein the second connection port is fluidly isolated from the liquid container.

16. An endoscope according to claim 15 further comprising means for pressurising the liquid container to cause liquid contained therein to flow from the liquid container to the first liquid flow channel via the first port.

17. An endoscope according to claim 10 wherein the connector comprises a body section having the first and second ports integrally formed therein.

18. A liquid supply system for an endoscope comprising:
a liquid receptacle, at least a portion of which is collapsible under the action of external pressure to pressurise the liquid contained therein;
an enclosure having an internal volume configured to house the liquid receptacle;
a fluid supply conduit in fluid communication with the internal volume of the enclosure for supplying a fluid thereto;
a liquid outlet conduit having a first end in fluid communication with the liquid receptacle and a second end for supplying liquid from the receptacle to an endoscope;
wherein the liquid receptacle and enclosure and arranged such that when a fluid is forced under pressure into the enclosure via the fluid supply conduit the resulting pressure rise within the enclosure causes pressurisation of the liquid contained within the receptacle.

19. A liquid supply system according to claim 18 wherein the liquid receptacle is a flexible bag.

20. A liquid supply system according to claim 18 wherein the enclosure comprises a closure member that is movable to an open configuration to permit the liquid receptacle to be inserted or removed from the enclosure.

21. A liquid supply system according to claim 18 wherein the fluid supply conduit and the liquid outlet conduit are concentric and enter the enclosure through a common sealed port.

22. A liquid supply system according to claim 21 wherein the fluid supply conduit and the liquid outlet conduit are fluidly isolated with the fluid supply conduit being open to the internal volume of the enclosure and the liquid outlet conduit being isolated from the internal volume of the enclosure and sealingly connected to the liquid receptacle.

23. A liquid supply system according to claim 22 comprising a sealing member configured to seal and locate the fluid supply and liquid outlet conduits relative to the enclosure, wherein the sealing member comprises a fluid pathway in fluid communication with the fluid supply conduit and the internal volume of the enclosure for fluidly connecting the fluid supply conduit to the internal volume of the enclosure.

24. A liquid supply system according to claim 18 wherein the fluid supply conduit and liquid outlet conduit are connected to a connector for connection to the connection block of an endoscope, by a connector for connecting the first end of the liquid flow channel to a liquid source, the connector comprising a first connection port configured for connection to an end of the liquid supply channel, a liquid source connection channel in fluid connection with the first port for connecting the first port to a liquid supply.

25. A liquid and fluid connection conduit for an endscope comprising:
a liquid conduit having a first end configured for connection to a liquid supply receptacle;
a concentric fluid conduit surrounding the liquid conduit and fluidly isolated therefrom having a first opening at an end corresponding to the first end of the liquid conduit;
a connector secured to the second ends of the liquid and fluid conduits, the connector comprising a first connection port configured for connection to the liquid supply channel, a liquid source connection channel in fluid connection with the first port for connecting the first port to a liquid source; and a second connection port configured for simultaneous connection to the second gas port, wherein the first connection port and the liquid source connection channel are fluidly isolated from the second connection port to prevent fluid communication between the second connection port and the liquid source.

26. A liquid and fluid connection conduit according to claim 25 comprising a second connector located at the opposing end of the conduits to the first connector for connecting the conduits to a pressurisation enclosure, the connector comprising a body through which the liquid conduit extends having and a liquid pathway fluidly connected to the fluid conduit opening to a distal surface of the body such that the fluid conduit is in open connection with the enclosure when the second connector is connected thereto.
